(19) United States
(12) Patent Application Publication
(10) Pub. No.: US 2005/0245898 A1
(43) Pub. Date: Nov. 3, 2005

(54) Surgical handpiece with quick-connection for irrigation and aspiration tubes

(76) Inventors: David Walter Wright, Littleton, CO (US); Lee William Travis, Littleton, CO (US)

Correspondence Address: Bausch & Lomb Incorporated, One Bausch & Lomb Place, Rochester, NY, 14604-2701, US

(21) Appl. No.: 10/834,633

(22) Filed: Apr. 29, 2004

(51) Int. Cl. A61M 25/16

(52) U.S. Cl. 604/533

(57) ABSTRACT

A surgical handpiece 10 for connection to an irrigation tube 14 and an aspiration tube 16 includes a keyed body 12 attached to each of the irrigation tube 14 and the aspiration tube 16. The surgical handpiece 10 includes structure 18 formed in the housing 20 for mating connection to the keyed body 12.

### BACKGROUND OF THE INVENTION

1. Field of the Invention

The present invention relates to ophthalmic surgical handpieces and associated tubing connections. More specifically, the present invention relates to a quick-connection assembly for quickly connecting and disconnecting irrigation and aspiration tubes from the surgical handpiece.

2. Description of Related Art

Present day ophthalmic surgical handpieces, such as phacoemulsification handpieces require connection to irrigation and aspiration tubing. The irrigation tubes supply irrigation fluid, such as balanced salt solution from a source to the distal end or tip of the surgical handpiece for flushing a surgical site with the irrigation fluid. Aspiration tubing is typically connected to an aspiration path within the surgical handpiece for transporting surgical fluids and tissue from the surgery site under the influence of an aspiration pump.

Obviously, it is very important that the proper line be connected to the proper port in the surgical handpiece. If the irrigation and aspiration tubes were to be inadvertently connected to the wrong ports, serious damage could occur to a patient's eye or, at the very least, unnecessarily delay surgery while correction is made. Typically, present day systems include separate and distinct markings on the tube to alert the user as to which tube is for which function. Such markings typically include a colored stripe running the length of one of the tubes. Separate and distinct connectors for each tube that are attached independently are also known.

These tubes are then press fit by hand onto the surgical handpiece ports by an operating room attendant. If the tubes are not properly press fit onto the surgical handpiece, the tubes may leak or become completely dislodged from the handpiece. This can result in unnecessary and unsanitary spilling of surgical fluids in the operating room or onto operating room staff or the patient.

Therefore, it would be highly desirable to have a device, which can be quickly and efficiently attached to a surgical handpiece so that the irrigation/aspiration tubes are properly connected to the surgical handpiece.

### BRIEF DESCRIPTION OF DRAWINGS

FIG. 1 is a perspective view of a surgical handpiece and quick connection device in accordance with the present invention;

FIG. 2 is an exploded perspective view of a quick connection device separated from a surgical handpiece in accordance with the present invention;

FIG. 3 is a side elevation view in accordance with the present invention showing a user connecting the quick connect device to a surgical handpiece;

FIG. 4 is a partial top view of a quick connect device connected to a surgical handpiece in accordance with the present invention;

FIG. 5 is a partial cut-away side elevation taken along line 5-5 of FIG. 4;

FIG. 6 is a partial top view of a quick connect device attached to a surgical handpiece in accordance with the present invention;

FIG. 7 is a partial side elevation view of an embodiment of the present invention taken along line 7-7 of FIG. 6;

FIG. 8 is a partial perspective view of an alternate embodiment of a quick connect device in accordance with the present invention;

FIG. 9 is an exploded partial perspective view of an alternate embodiment of a quick-connect device in accordance with the present invention;

FIG. 10 is a top view of the device of FIG. 9 connected to a surgical handpiece; and

FIG. 11 is a partial side elevation view of yet another alternate embodiment of a quick-connect device in accordance with the present invention.

### DETAILED DESCRIPTION OF THE PREFERRED EMBODIMENT

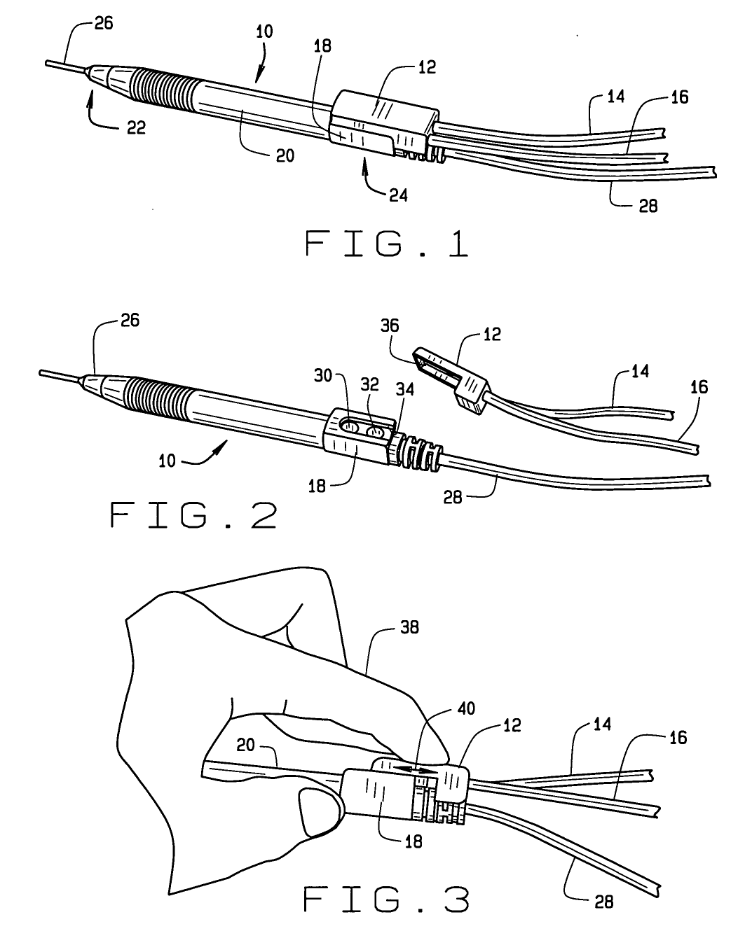

FIG. 1 shows a surgical handpiece 10 connected to a quick connect device 12, in accordance with the present invention. Quick connect device 12 has a keyed body attached to each of an irrigation line 14 and an aspiration line or tube 16. The keyed body 12 is attached to a mating structure shown generally at 18, on surgical handpiece 10.

Surgical handpiece 10 includes an elongated housing 20 having a distal end, shown generally at 22, and a proximal end, shown generally at 24. A surgical device 26 is attached to distal end 22 for performing a surgical function. Structure 18 is formed in the housing 20 for mating connection to the keyed body 12, which is attached to each of the irrigation tube 14 and the aspiration tube 16. In addition, a power cord 28 is attached to surgical handpiece 10.

It is noted that while FIG. 1 shows surgical handpiece 10 as a phacoemulsification handpiece with a sleeve and needle 26 attached to distal end 22, other surgical handpieces with other surgical devices 26 attached thereto may be used and are within the scope of the present invention. For instance, surgical handpiece 10 could be a vitrectomy cutter, a pneumatic scissors, or simply an irrigation/aspiration handpiece.

As it is shown in FIG. 1, an additional important feature is that keyed body 12 be constructed and formed such that keyed body 12 may only be mated onto the surgical handpiece 10 in one orientation. This is to ensure proper connection of the irrigation and aspiration tubes 14 and 16 to the handpiece 10. In this way, a safe and proper connection of the tubes to the handpiece may be assured. It will be appreciated that other types of keyed connections may be made, such as twist-on bayonet type connections that provide some tactile feedback to the user to indicate that a proper connection has been made.

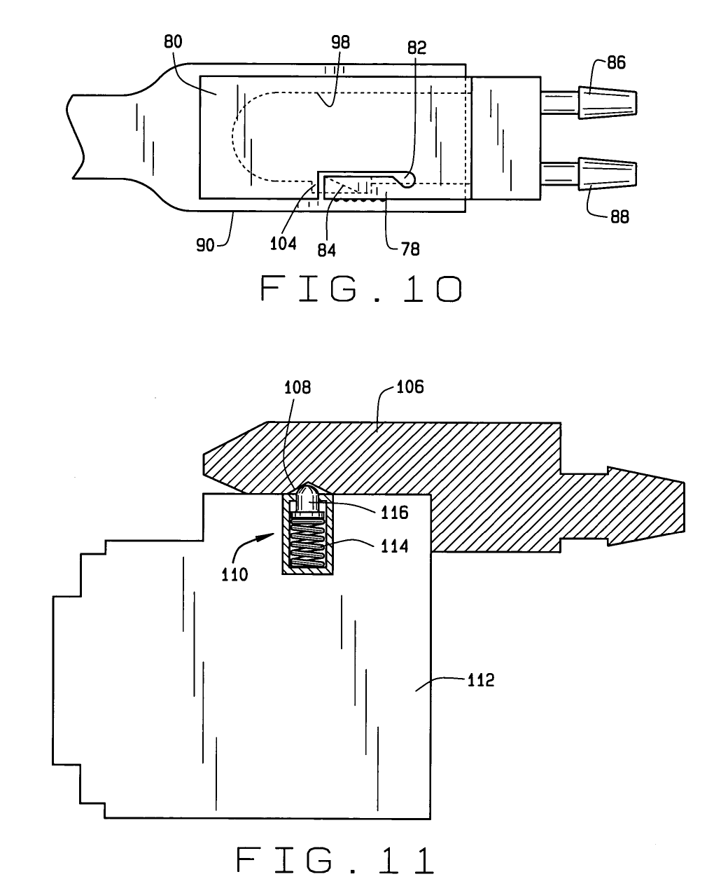

Yet another inventive element that is preferably incorporated into keyed body 12, is a detent formed on the keyed body 12 for providing a user with tactile feedback confirmation that the keyed body 12 is attached to the surgical handpiece 10. Two embodiments of the detent detail are shown below at FIGS. 9-11; however, those skilled in the art will readily appreciate that such a detent may take the form of several structures, such as a rib or other protrusion formed on body 12 that slidingly locks or “clicks” into place in a corresponding slot or indentation in surgical handpiece 10.

FIG. 2 shows surgical handpiece 10 detached from keyed body 12. As can be seen, the mating structure 18 shows an irrigation port 30 and an aspiration port 32, which are to be connected to tubes 14 and 16 respectively. Tube 14 is connected to an irrigation source, such as a balanced salt solution or other irrigation fluids. Tube 16 is connected to an aspiration collection source, such as a bag or cassette (not shown), which is received through port 32 and transported through tube 16 to such collection bag or cassette.

Mating structure 18 preferably includes a slot defined by wall 34 into which tab 36 matingly slides.

FIG. 3 shows a side elevation view of a user 38 slidingly attaching and detaching keyed body 12 to mating structure 18, as indicated by arrowed line 40.

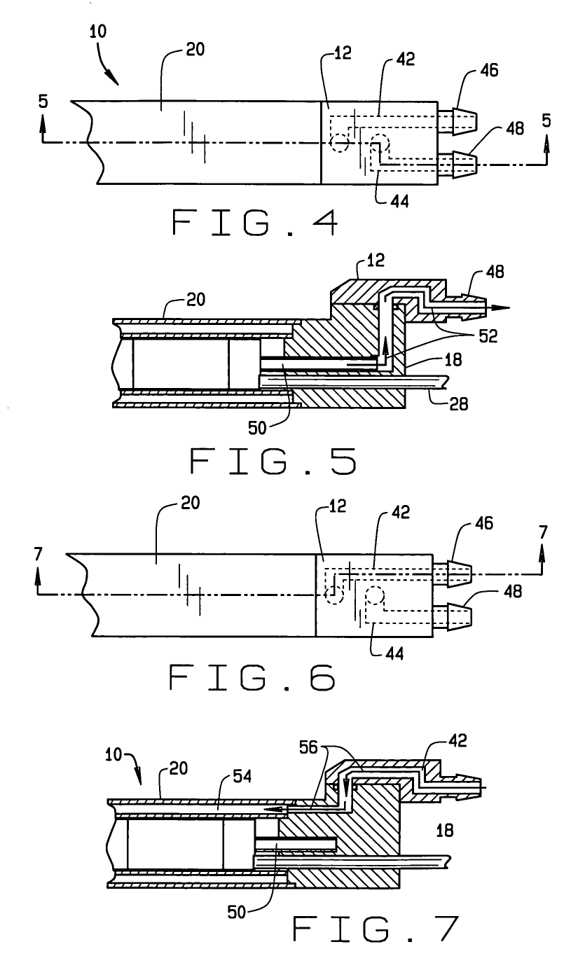

FIG. 4 is a partial top view of surgical handpiece 10 attached to keyed body 12, wherein dashed lines 42 and 44 indicate internal paths formed in keyed body 12 for allowing irrigation fluids to flow into and aspiration fluids out of keyed body 12. FIG. 4 also shows irrigation barb or port 46 and aspiration port 48 which are to be connected to irrigation tube 14 and aspiration tube 16 respectively in manufacture.

FIG. 5 is a partial side elevation view of FIG. 4 taken along line 5-5 of FIG. 4. FIG. 5 shows keyed body 12 attached to mating structure 18, which includes an aspiration path 50 through which aspiration fluids flow in the direction of arrows 52.

FIG. 6 is a view similar to that of FIG. 4.

FIG. 7 is a partial side elevation view of FIG. 6 taken along line 7-7. FIG. 7 shows keyed body 12 connected to handpiece 10 and elongated body 20 along with the irrigation path 42 and body 12 connected to irrigation path 54 of surgical handpiece 10 through which irrigation fluid is fed in the direction of arrows 56.

In this way, an inventive surgical handpiece has been shown incorporating a quick connect device for ensuring that proper, quick reliable connections are made between irrigation and aspiration tubes and the surgical handpiece.

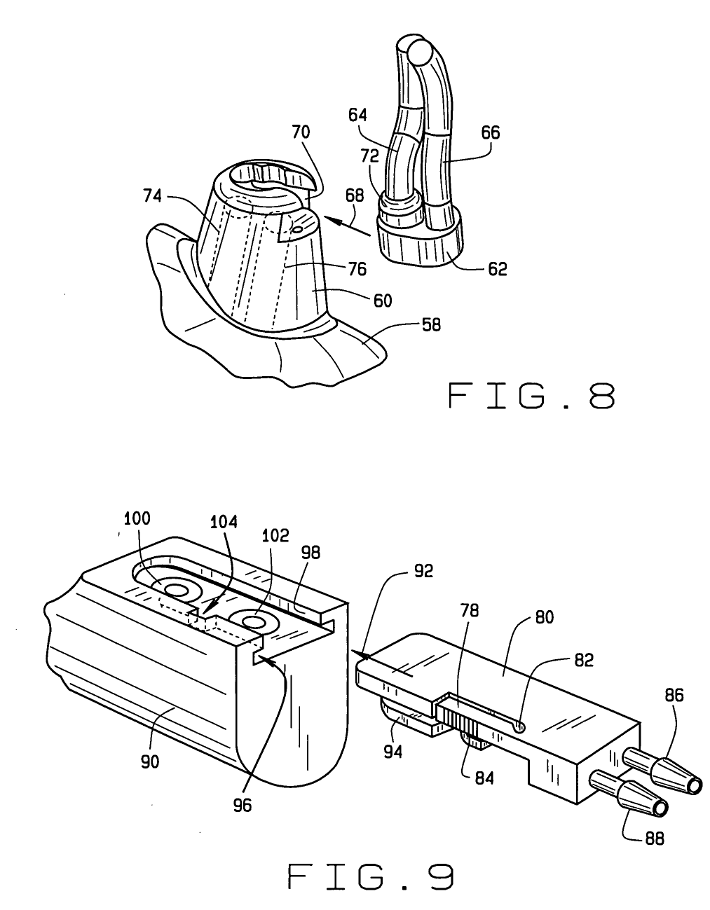

FIG. 8 discloses a partial perspective view of an alternate embodiment of a surgical handpiece in accordance with the present invention. FIG. 8 includes a surgical handpiece body partially shown at 58 and having mating structure 60 for quick connection to keyed body 62. Attached to body 62 are irrigation tube 64 and aspiration tube 66. Keyed body 62 fits into mating structure 60 as shown by arrow 68 and fits into the slot shown generally at 70. As can be seen, body 62 is keyed by collar 72 associated with irrigation tube 64. Once body 62 is attached to mating structure 60, irrigation fluid is fed through tube 64 into irrigation path 74 (shown as dashed lines). Aspiration tissue and fluids are fed through aspiration path 76 into tube 66 and on to a collection bag or other collection reservoirs (not shown).

FIG. 9 shows a partial exploded perspective view of an alternate embodiment in accordance with the present invention. The embodiment of FIG. 9 is similar to the embodiment shown above, with the addition of a detent arm 78 formed in keyed body 80. Detent arm 78 is defined by the slot or groove 82 formed in the keyed body 80. Detent arm 78 includes a knob 84. Keyed body 80 includes barbs 86 and 88 for attachment to irrigation/aspiration tubes, as described above.

Keyed body 80 slidingly attaches to mating structure 90 of a handpiece, by insertion in the direction of arrow 92. A flange 94 of keyed body 80 rides within a slot shown generally at 96 and fits in within the wall defined at 98. Passageways (not shown) associated with barbs 86 and 88 align with ports 100 and 102 in a fashion similar to that described above with respect to other embodiments. Knob 84 then “clicks” into notch 104 when keyed body 80 is fully inserted onto mating structure 90.

Detent arm 78 and knob 84 cooperate with notch 104 to provide feedback and assurance to a user that keyed body 80 is properly fitted onto mating structure 90. It may be desirable to form detent arm 78, such that detent arm is a ‘break-away’ arm, i.e. arm 78 only functions properly for a limited number or even just one use. This would help insure that users dispose of the keyed body 80 and associated tubing without use on additional patients.

FIG. 10 is a top view of keyed body 80 attached to mating structure 90. Wall 98 is shown as a dashed line, as is notch 104 and knob 84.

FIG. 11 shows yet another alternate embodiment where a keyed body 106, similar to the keyed bodies describe above, includes a groove or indentation 108, which receives a detent shown generally at 110, to provide a user with feedback to indicate that keyed body 106 has been properly inserted into mating structure 112. Detent 110 includes a spring 114 and a pin 116, which are received within slot 108.

It will be appreciated that the two different detent embodiments are for illustration purposes and other detent structures may be used for connection of a keyed body to the mating structure and still be within the scope of the present invention.

Thus, there has been shown an inventive quick-connect device and associated surgical handpiece for providing quick-connection of surgical tubes to a surgical handpiece. It will be appreciated that various other embodiments may be formed and still be within the scope of the present invention.

1. A quick-connect device for connecting irrigation and aspiration tubes to a surgical handpiece comprising:
a keyed body attached to each of an irrigation tube and an aspiration tube; and
wherein the keyed body is to be attached to a mating structure on a surgical handpiece.

2. The invention of claim 1, wherein the keyed body may only be mated to the surgical handpiece in one orientation to ensure proper connection to the handpiece.

3. The invention of claim 1 further including a detent formed on the keyed body for providing a user with tactile feedback confirmation that the body is attached to the surgical handpiece.

4. A surgical handpiece for connection to irrigation and aspiration tubes comprising:
an elongated housing having a distal end and a proximal end;
a surgical device attached to the distal end for performing a surgical function; and
structured formed in the housing for mating connection to a keyed body attached to each of an irrigation tube and an aspiration tube.

5. The invention of claim 4, wherein the keyed body may only be mated to the surgical handpiece in one orientation to ensure proper connection to the handpiece.

6. The invention of claim 4 further including structure for receiving a detent on the keyed body.

7. A surgical handpiece system for connecting irrigation and aspiration tubes to the surgical handpiece comprising:
a keyed body attached to each of an irrigation tube and an aspiration tube;
an elongated surgical handpiece housing having a distal end and a proximal end;
a surgical device attached to the distal end for performing a surgical function; and
structure formed in the housing for mating connection to the keyed body thereby connecting the irrigation and aspiration tubes to the surgical handpiece.

8. The invention of claim 7, wherein the keyed body may only be mated to the surgical handpiece in one orientation to ensure proper connection to the handpiece.

9. The invention of claim 7 further including a detent formed on the keyed body for providing a user with tactile feedback confirmation that the body is attached to the surgical handpiece.
